(19) United States
(12) Patent Application Publication
(10) Pub. No.: US 2011/0239743 A1
(43) Pub. Date: Oct. 6, 2011

(54) VEHICLE WORKING-FLUID EVALUATING SYSTEM

(75) Inventors: Satoshi YAMASHITA, Wako (JP); Takeshi Kurata, Wako (JP); Yoshiteru Matsuura, Wako (JP)

(73) Assignee: HONDA MOTOR CO., LTD., Tokyo (JP)

(21) Appl. No.: 13/021,786

(22) Filed: Feb. 7, 2011

(30) Foreign Application Priority Data
Mar. 31, 2010 (JP) 2010-082132

(51) Int. Cl. G01N 33/30

(52) U.S. Cl. 73/53.05

(57) ABSTRACT

A vehicle working-fluid evaluating system includes an obtaining device configured to obtain a plurality of deterioration elements related to deterioration factors that are generated as a vehicle travels and that accelerate deterioration of working fluid. A deterioration-level calculator is configured to calculate, based on the plurality of deterioration elements obtained by the obtaining device, an oil deterioration estimate. A severity determining device is configured to determine, in accordance with the oil deterioration estimate calculated by the deterioration-level calculator, severity of usage of the working fluid for a transmission mounted on the vehicle. A change-timing determining device is configured to determine the working-fluid change timing in accordance with the severity of usage of the working fluid determined by the severity determining device. A notification device is configured to indicate the determined working-fluid change timing determined by the change-timing determining device.

### CROSS-REFERENCE TO RELATED APPLICATIONS

The present application claims priority under 35 U.S.C. §119 to Japanese Patent Application No. 2010-082132, filed in the Japan Patent Office on Mar. 31, 2010, entitled “VEHICLE WORKING-FLUID EVALUATING SYSTEM”. The contents of this application are incorporated herein by reference in their entirety.

### BACKGROUND OF THE INVENTION

1. Field of the Invention

The present invention relates to a vehicle working-fluid evaluating system.

2. Description of the Related Art

In general, working fluid used for lubricating an automatic transmission mounted on a vehicle (automatic transmission fluid (ATF) or may be simply referred to as oil) deteriorates with time. Therefore, it is necessary to change oil at appropriate times. For this, a certain change criterion, such as a cumulative travel distance of the vehicle or elapsed time, which serves as a certain measure for determining when to change oil is determined in advance. When such a change criterion is met, notification indicating that oil is to be changed is provided to the user of the vehicle, on the assumption that the oil has deteriorated. In this case, to vehicles with the same travel distance or elapsed time, notification of oil change is provided at exactly the same timing.

In practice, however, the level of deterioration (deterioration level) of working fluid varies depending on how the user drives (or uses) the vehicle. In other words, the deterioration level of working fluid varies depending on severity of usage of the working fluid. Therefore, making notification of oil change on the basis only of the travel distance or elapsed time may cause inconvenience. For example, even when oil deterioration caused by user's rough driving already affects the lubricating action of the oil, the notification of oil change may not be made if the change criterion has not been met. Conversely, for example, even when oil has not actually deteriorated and can still be used because of user's gentle driving, the notification of oil change may be made if the change criterion has been met.

Japanese Unexamined Patent Application Publication No. 2006-234155 discloses a clutch evaluating system that evaluates severity of usage of a clutch. With this clutch evaluating system, it is possible to estimate the deterioration level of working fluid. In the technique described in Japanese Unexamined Patent Application Publication No. 2006-234155, the severity of usage of the clutch is evaluated based on an ON/OFF state of a gear. The working fluid deteriorates as the severity of usage of the clutch increases. That is, the level of deterioration of the working fluid varies depending on the usage of the clutch. Therefore, notification of oil change may be made in accordance with the severity of usage of the clutch.

As described above, the level of deterioration of the working fluid cannot be determined simply based on the travel distance or elapsed time. The level of deterioration of the working fluid depends on how the user uses the vehicle, in other words, depends on severity of usage of the working fluid (i.e., the user's severity to the working fluid). Specifically, depending on how the vehicle is driven, deterioration of working fluid is accelerated by various deterioration factors, such as shear caused by rotation of a rotational body such as a gear inside the transmission, oxidation of the working fluid caused by an increase in temperature of the working fluid (oil temperature), and occurrence of foreign bodies caused by wear in the gear or transmission case. Therefore, it is not appropriate to directly apply the technique of the clutch evaluating system disclosed in Japanese Unexamined Patent Application Publication No. 2006-234155 to a working-fluid evaluating system. In the clutch evaluating system described above, only an ON/OFF state of a gear (i.e., shear caused by rotation of a rotational body such as a gear inside a transmission) is considered as a deterioration factor, and no other deterioration factors are taken into account.

### SUMMARY OF THE INVENTION

According to one aspect of the present, a vehicle working-fluid evaluating system indicates working-fluid change timing when working fluid for a transmission mounted on a vehicle is changed. The vehicle working-fluid evaluating system includes an obtaining device, a deterioration-level calculator, a severity determining device, a change-timing determining device, and a notification device. The obtaining device is configured to obtain a plurality of deterioration elements related to deterioration factors that are generated as the vehicle travels and that accelerate deterioration of the working fluid. The deterioration-level calculator is configured to calculate, based on the plurality of deterioration elements obtained by the obtaining device, an oil deterioration estimate corresponding to a level of deterioration of the working fluid for each of the plurality of deterioration elements. The severity determining device is configured to determine, in accordance with the oil deterioration estimate calculated by the deterioration-level calculator, severity of usage of the working fluid for the transmission mounted on the vehicle. The change-timing determining device is configured to determine the working-fluid change timing in accordance with the severity of usage of the working fluid determined by the severity determining device. The notification device is configured to indicate the determined working-fluid change timing determined by the change-timing determining device.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 is a block diagram schematically illustrating an overall configuration of a vehicle working-fluid evaluating system according to an embodiment of the present invention;

FIG. 2 is a schematic view illustrating an embodiment of a power transmission configuration of an automatic transmission mounted on a vehicle;

FIG. 3 is a block diagram schematically illustrating a vehicle-mounted terminal;

FIG. 4 is a flowchart illustrating an embodiment of an oil change notification process;

FIG. 5 is a flowchart illustrating an embodiment of a vehicle-speed deterioration-level calculating process;

FIG. 6 is a characteristic diagram illustrating a relationship between vehicle speed and deterioration coefficient;

FIG. 7 is a conceptual diagram illustrating driving time in each vehicle speed range;

FIG. 8 is a flowchart illustrating an embodiment of a creep deterioration-level calculating process;

FIG. 9 is a characteristic diagram illustrating a relationship between creep torque and deterioration coefficient;

FIG. 10 is a conceptual diagram illustrating creep time for each level of creep torque;

FIG. 11 is a flowchart illustrating an embodiment of a belt-displacement deterioration-level calculating process;

FIG. 12 is a flowchart illustrating an embodiment of a starting deterioration-level calculating process;

FIG. 13 is a characteristic diagram illustrating a relationship between starting energy and deterioration coefficient;

FIG. 14 is a conceptual diagram illustrating the number of starting operations for each level of starting energy;

FIG. 15 is a flowchart illustrating an embodiment of an SC-work deterioration-level calculating process;

FIG. 16 is a conceptual diagram illustrating total SC work;

FIG. 17 is a flowchart illustrating an embodiment of an oil-temperature deterioration-level calculating process;

FIG. 18 is a characteristic diagram illustrating a relationship between oil temperature and deterioration coefficient; and

FIG. 19 is a conceptual diagram illustrating driving time in each oil temperature range.

### DESCRIPTION OF THE EMBODIMENTS

Embodiments of the present invention will now be described in detail with reference to the attached drawings, wherein like reference numerals designate corresponding or identical elements throughout the various drawings.

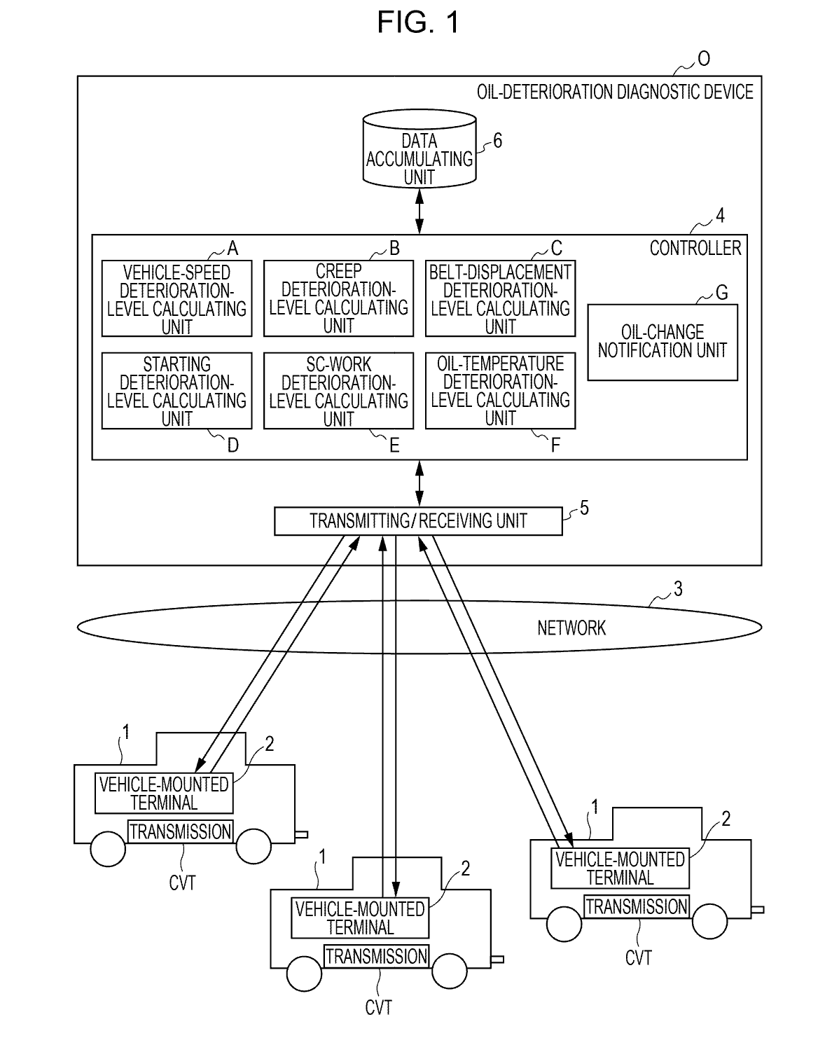

FIG. 1 is a block diagram schematically illustrating an overall configuration of a vehicle working-fluid evaluating system according to the embodiment of the present invention. As illustrated in FIG. 1, the working-fluid evaluating system includes vehicle-mounted terminals 2 mounted on respective vehicles 1, and an oil-deterioration diagnostic device O located, for example, at a system management company that provides the working-fluid evaluating system. Each vehicle-mounted terminal 2 and the oil-deterioration diagnostic device O are connected such that they can transmit and receive various types of data to and from each other via a predetermined communication network 3, such as a packet communication network using a mobile phone line or a wireless local area network (LAN).

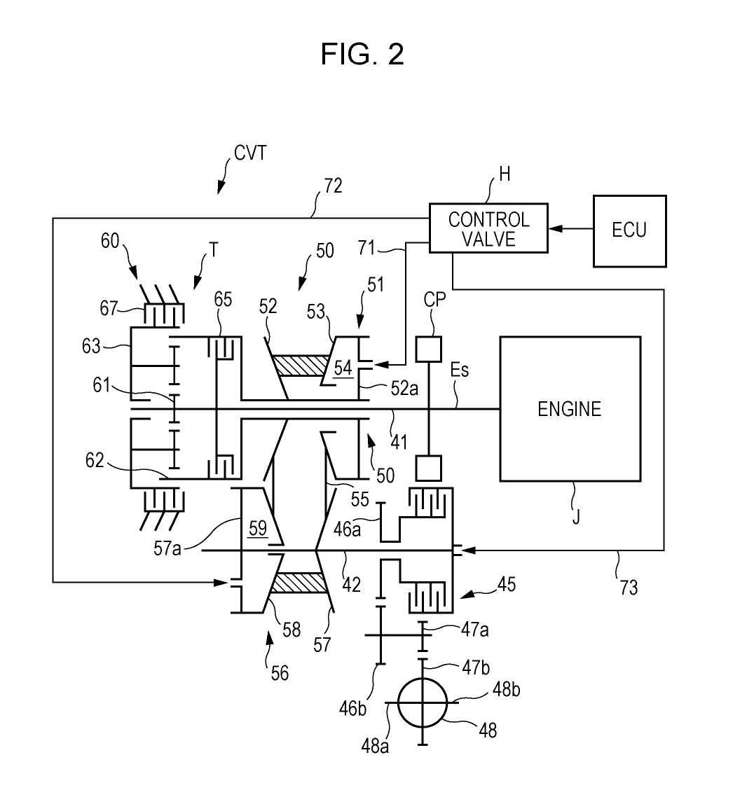

The vehicle-mounted terminal 2 has basic functions of a computer. The vehicle-mounted terminal 2 obtains, one by one, various types of data (referred to as deterioration element data) related to deterioration elements which may accelerate deterioration of working fluid (ATF) circulating within an automatic transmission CVT (e.g., a continuously variable transmission illustrated in FIG. 2 described below) mounted on the vehicle 1. Examples of such deterioration elements include vehicle speed, creep force (creep torque), belt displacement, starting energy (starting force), start clutch (SC) work, and oil temperature which occur as the vehicle 1 travels and may cause various deterioration factors. The obtained deterioration element data is transmitted to the oil-deterioration diagnostic device O. The oil-deterioration diagnostic device O may transmit oil change timing to the vehicle-mounted terminal 2. In such a case, the user can be notified of the oil change timing, for example, displayed on a display or presented by voice guidance.

The oil-deterioration diagnostic device O has basic functions of a server. Based on the deterioration element data transmitted one by one from the vehicle-mounted terminal 2 mounted on each vehicle 1, the oil-deterioration diagnostic device O calculates a deterioration level for each of various deterioration factors. Based on the calculated deterioration level, the oil-deterioration diagnostic device O evaluates, for each user (vehicle), severity of usage of the working fluid (i.e., the user's severity to the working fluid) that lubricates the automatic transmission CVT. The deterioration level is an “oil deterioration estimate per unit distance” which depends on how each vehicle 1 is driven by the user. From the deterioration element data obtained from the vehicle 1, the deterioration level can be calculated for each deterioration factor in accordance with a predetermined arithmetic expression (described in detail below).

Based on the severity of usage of the working fluid evaluated for each vehicle 1, the oil-deterioration diagnostic device O determines oil change timing for the vehicle 1. The oil-deterioration diagnostic device O transmits the determined oil change timing to vehicles 1 for which the determined oil change timing will be reached within a predetermined period of time (e.g., one month). Alternatively, regardless of the predetermined period of time, the oil-deterioration diagnostic device O may transmit oil change timing to all vehicles 1 upon determination of the oil change timing. Alternatively, vehicle mechanics or dealers may be able to specify appropriate vehicles 1 to which oil change timing is to be transmitted.

FIG. 2 illustrates an example of the automatic transmission CVT for which severity of usage of working fluid is evaluated in the vehicle working-fluid evaluating system according to the embodiment of the present invention. FIG. 2 is a schematic view illustrating an embodiment of a power transmission configuration of the automatic transmission CVT mounted on the vehicle 1.

As can be seen from FIG. 2, the automatic transmission CVT according to the present embodiment includes an engine J and a continuously variable transmission mechanism T coupled via a coupling mechanism CP to an output shaft Es of the engine J. The continuously variable transmission mechanism T includes a metal V-belt mechanism 50 disposed between an input shaft 41 and a counter shaft 42, a forward/backward movement switching mechanism 60 disposed on the input shaft 41, and a start clutch 45 disposed on the counter shaft 42. The continuously variable transmission mechanism T is intended for used in a vehicle. The input shaft 41 is coupled via the coupling mechanism CP to the output shaft Es of the engine J. A driving force from the start clutch 45 is transmitted from a differential mechanism 48 via left and right axle shafts 48a and 48b to left and right wheels (not shown).

The metal V-belt mechanism 50 includes a driving-side movable pulley 51 disposed on the input shaft 41, a driven-side movable pulley 56 disposed on the counter shaft 42, and a metal V-belt 55 wound around the pulleys 51 and 56. The driving-side movable pulley 51 includes a fixed pulley half 52 rotatably disposed on the input shaft 41 and a movable pulley half 53 movable relative to the fixed pulley half 52 in the axial direction of the input shaft 41. A driving-side cylinder chamber 54 surrounded by a cylinder wall 52a is formed to one side of the movable pulley half 53. A pulley-control hydraulic pressure supplied from a control valve H through an oil passage 71 to the driving-side cylinder chamber 54 generates a driving-side pressure that moves the movable pulley half 53 in the axial direction of the input shaft 41. Similarly, the driven-side movable pulley 56 includes a fixed pulley half 57 rotatably disposed on the counter shaft 42 and a movable pulley half 58 movable relative to the fixed pulley half 57 in the axial direction of the counter shaft 42. A driven-side cylinder chamber 59 surrounded by a cylinder wall 57a is formed to one side of the movable pulley half 58. A pulley-control hydraulic pressure supplied from the control valve H through an oil passage 72 to the driven-side cylinder chamber 59 generates a driven-side pressure that moves the movable pulley half 58 in the axial direction of the counter shaft 42.

As can be seen from the configuration described above, the supply of hydraulic pressures (driving-side and driven-side pressures) to the cylinder chambers 54 and 59 can be controlled by the control valve H. When the driving-side and driven-side pressures are different, the winding radius of the metal V-belt 55 changes in accordance with a change in groove width of the pulleys 51 and 56. It is thus possible to steplessly change the gear ratio.

The forward/backward movement switching mechanism 60 is a planetary gear mechanism. The forward/backward movement switching mechanism 60 includes a sun gear 61 combined with the input shaft 41, a ring gear 62 combined with the fixed pulley half 52, a carrier 63 that can be held in position by a backward brake 67, and a forward clutch 65 that can couple the sun gear 61 to the ring gear 62. In the forward/backward movement switching mechanism 60, when the forward clutch 65 is in engagement, all the gears 61, 62, and 63 rotate integrally with the input shaft 41, and the driving-side movable pulley 51 is rotated by the engine J in the direction of rotation of the input shaft 41 (i.e., in the forward direction). On the other hand, when the backward brake 67 is in engagement, since the carrier 63 is held in position, the ring gear 62 is driven in the direction opposite the movement of the sun gear 61, while the driving-side movable pulley 51 is rotated in the direction opposite the rotation of the input shaft 41 (i.e., in the backward direction).

The start clutch 45 is a clutch that controls power transmission between the counter shaft 42 and output-side members, which are power transmission gears 46a, 46b, 47a, and 47b. When the counter shaft 42 engages with the power transmission gears 46a, 46b, 47a, and 47b, power transmission between them can be made possible. Therefore, when the start clutch 45 is in engagement, a gear-changed engine output is transmitted by the metal V-belt mechanism 50 via the power transmission gears 46a, 46b, 47a, and 47b to the differential mechanism 48, which divides the engine output. The resulting engine outputs are transmitted via the left and right axle shafts 48a and 48b to the left and right wheels (not shown). This engagement control of the start clutch 45 is done by a clutch-control hydraulic pressure supplied from the control valve H through an oil passage 73.

Referring back to FIG. 1, the oil-deterioration diagnostic device O and the vehicle-mounted terminal 2 that perform the above-described various control operations will be described in detail. As illustrated in FIG. 1, the oil-deterioration diagnostic device O includes a controller 4, a transmitting/receiving unit 5, and a data accumulating unit 6. The controller 4 is, for example, a microcomputer that includes a central processing unit (CPU), a read-only memory (ROM), a random-access memory (RAM), and an input/output interface (not shown) and realizes predetermined functions in accordance with various control programs stored in the ROM while using a temporary memory function of the RAM. In the present embodiment, the controller 4 includes a vehicle-speed deterioration-level calculating unit A, a creep deterioration-level calculating unit B, a belt-displacement deterioration-level calculating unit C, a starting deterioration-level calculating unit D, a start clutch (SC)-work deterioration-level calculating unit E, an oil-temperature deterioration-level calculating unit F, and an oil-change notification unit G.

The vehicle-speed deterioration-level calculating unit A calculates a vehicle-speed deterioration level based on data related to vehicle travel speed, the data being part of the deterioration element data transmitted from the vehicle-mounted terminal 2. The vehicle-speed deterioration level corresponds to a working-fluid deterioration level related to vehicle speed which can be a factor that deteriorates working fluid. The creep deterioration-level calculating unit B calculates a creep deterioration level based on data related to driving force (creep torque) applied at the time of creep phenomenon, the data being part of the deterioration element data transmitted from the vehicle-mounted terminal 2. The creep deterioration level corresponds to a working-fluid deterioration level related to creep torque which can be a factor that deteriorates working fluid. The belt-displacement deterioration-level calculating unit C calculates a belt-displacement deterioration level based on data related to winding radius of the metal V-belt 55 (or belt winding diameter) that changes as the gear ratio changes, the data being part of the deterioration element data transmitted from the vehicle-mounted terminal 2. The belt-displacement deterioration level corresponds to a working-fluid deterioration level related to a gear change operation which can be a factor that deteriorates working fluid.

The starting deterioration-level calculating unit D calculates a starting deterioration level based on data related to starting force applied for starting the vehicle 1 (e.g., the more sudden the starting, the larger the starting force), the data being part of the deterioration element data transmitted from the vehicle-mounted terminal 2. The starting deterioration level corresponds to a working-fluid deterioration level related to sudden starting which can be a factor that deteriorates working fluid. The SC-work deterioration-level calculating unit E calculates an SC-work deterioration level based on data related to the amount of work of the start clutch 45 applied for starting the vehicle 1 (e.g., the more sudden the starting, the larger the amount of work), the data being part of the deterioration element data transmitted from the vehicle-mounted terminal 2. The SC-work deterioration level corresponds to a working-fluid deterioration level related to sudden starting which can be a factor that deteriorates working fluid. The oil-temperature deterioration-level calculating unit F calculates an oil-temperature deterioration level based on data related to oil temperature of working fluid, the data being part of the deterioration element data transmitted from the vehicle-mounted terminal 2. The oil-temperature deterioration level corresponds to a working-fluid deterioration level related to oil temperature which can be a factor that deteriorates working fluid. The oil-change notification unit G evaluates the severity of usage of the working fluid (i.e., the user's severity to the working fluid) based on each of the calculated deterioration levels, determines oil change timing for users with severe usage, and outputs the determined oil change timing to the transmitting/receiving unit 5.

The transmitting/receiving unit 5 transmits and receives various types of data to and from the vehicle-mounted terminals 2 via the communication network 3. When deterioration element data is received from the vehicle-mounted terminal 2 of each vehicle 1, the transmitting/receiving unit 5 accumulates and stores the received deterioration element data in the data accumulating unit 6. When oil change timing is output from the oil-change notification unit G as described above, the transmitting/receiving unit 5 transmits the output oil change timing to the vehicle-mounted terminal 2 of each appropriate vehicle 1. In the data accumulating unit 6, information (e.g., vehicle number) for identifying each vehicle 1 to be notified of oil change timing by the oil-deterioration diagnostic device O is registered in advance. The obtained deterioration element data stored in the data accumulating unit 6 is associated with the vehicles 1 from which the deterioration element data is supplied.

The data accumulating unit 6 may store, in advance, an arithmetic expression used to calculate a deterioration level for each deterioration factor based on predetermined deterioration element data obtained from the vehicle 1, the deterioration level indicating severity of usage of working fluid. As will be described below, in the present embodiment, such arithmetic expressions are used to calculate deterioration levels for respective deterioration factors which may deteriorate working fluid circulating within the continuously variable transmission CVT such as that illustrated in FIG. 2. Examples of the deterioration levels include a vehicle-speed deterioration level, a creep deterioration level, a belt-displacement deterioration level, a starting deterioration level, an SC-work deterioration level, and an oil-temperature deterioration level.

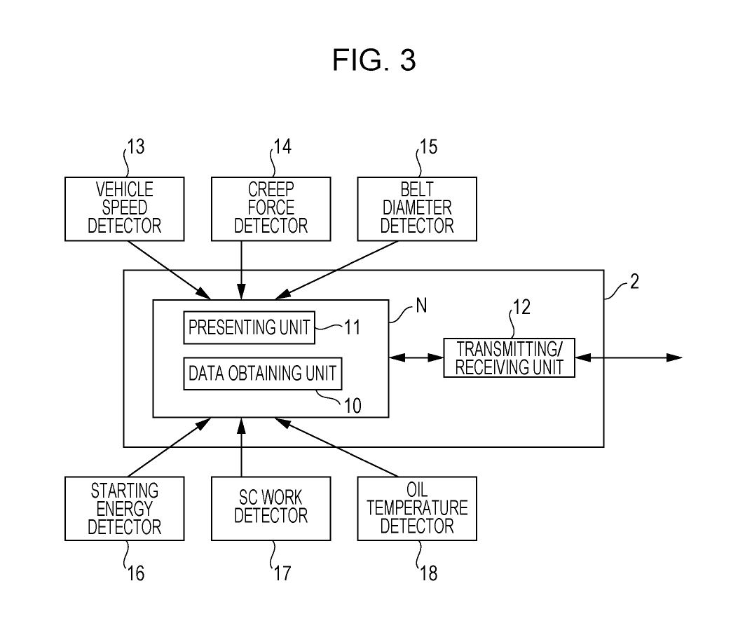

As illustrated in FIG. 3, each vehicle 1 includes not only the vehicle-mounted terminal 2 but also a vehicle speed detector 13, a creep force detector 14, a belt diameter detector 15, a starting energy detector 16, an SC work detector 17, and an oil temperature detector 18 each detecting a different type of deterioration element data. The vehicle speed detector 13 detects a vehicle speed of the vehicle 1. The creep force detector 14 detects a driving force (creep torque) applied at the time of creep phenomenon in which the vehicle 1 moves with the engine at idle, with the accelerator pedal not being depressed by the driver. The creep force detector 14 also detects the corresponding creep time. The belt diameter detector 15 detects the amount of movement (displacement) of the winding radius of the metal V-belt 55 at the movable pulley half 53 as the gear ratio changes.

The starting energy detector 16 detects a driving force (starting force) applied for starting a stopped vehicle 1. The SC work detector 17 detects the amount of work of the start clutch 45 applied for starting a stopped vehicle 1. The oil temperature detector 18 detects a temperature of working fluid. A measuring unit (not shown) measures the length of driving time from the start to end of driving of the vehicle 1, and also measures a cumulative value of distance traveled (i.e., cumulative travel distance).

The vehicle-mounted terminal 2 includes a controller N and a transmitting/receiving unit 12. The transmitting/receiving unit 12 transmits and receives various types of data to and from the oil-deterioration diagnostic device O via the communication network 3. The controller N is constituted by a CPU and includes a data obtaining unit 10 and a presenting unit 11. The data obtaining unit 10 obtains, one by one, the deterioration element data detected by the vehicle speed detector 13, the creep force detector 14, the belt diameter detector 15, the starting energy detector 16, the SC work detector 17, and the oil temperature detector 18 and the driving time and cumulative travel distance measured by the measuring unit. Then, the data obtaining unit 10 transmits the obtained data from the transmitting/receiving unit 12 to the oil-deterioration diagnostic device O.

When oil change timing is received from the oil-deterioration diagnostic device O, the data obtaining unit 10 outputs the received oil change timing to the presenting unit 11. The presenting unit 11 may include a display (display device) that displays the oil change timing or an audio device that reads the oil change timing out loud. For example, a message such as “Please change oil before traveling another 100 km” or “Please change oil within 2 weeks” is presented to notify the user of the oil change timing. The travel distance or time before the oil change can be estimated based on how the vehicle 1 has been driven. The user can thus be notified of when to change oil by viewing or listening to information about the oil change timing presented by the presenting unit 11.

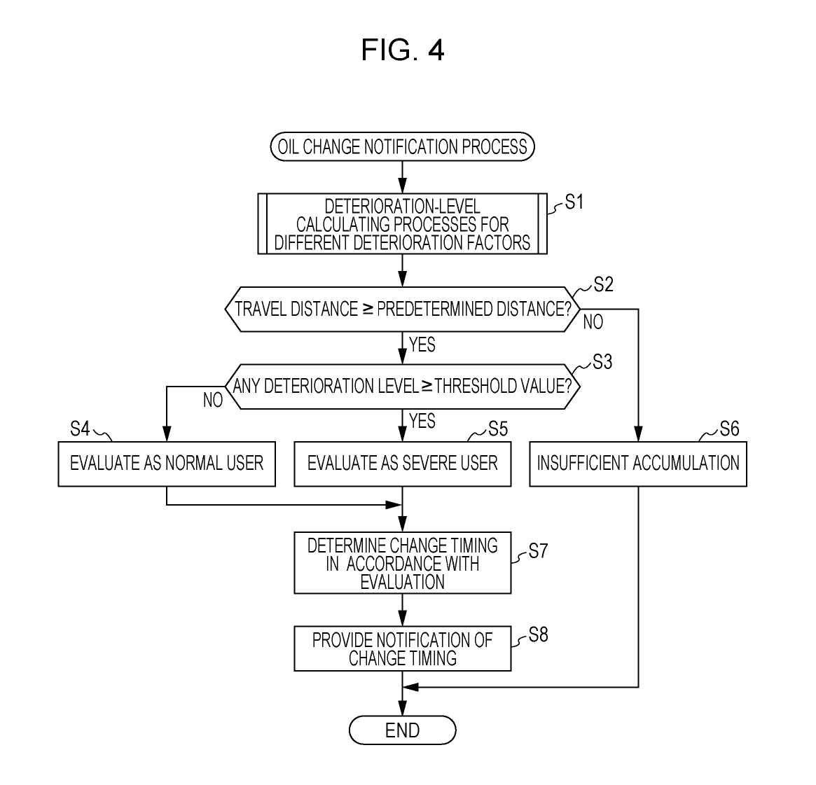

Next, a description will be given of an “oil change notification process” which involves evaluating severity of usage of working fluid for each of various deterioration factors, and notifying each user (vehicle) of appropriate oil change timing. FIG. 4 is a flowchart illustrating an embodiment of an oil change notification process. This process is executed by the controller 4 of the oil-deterioration diagnostic device O. The controller 4 may execute this process automatically at predetermined time intervals or when a notification request (execution command) is received from each vehicle 1. It is to be understood that this process is performed for each vehicle 1.

In step S1, “deterioration-level calculating processes for different deterioration factors” are executed. The processes involve calculating deterioration levels (oil deterioration estimates) for respective deterioration factors, such as a vehicle-speed deterioration level, a creep deterioration level, a belt-displacement deterioration level, a starting deterioration level, an SC-work deterioration level, and an oil-temperature deterioration level. The “deterioration-level calculating processes for different deterioration factors” will now be described in detail. Each deterioration factor requires a different algorithm for calculating a deterioration level. Therefore, the following describes a process for each of various types of related data which may be deterioration factors. That is, the following describes a process for each of deterioration elements such as vehicle speed, creep force, belt displacement, starting energy, SC work, and oil temperature.

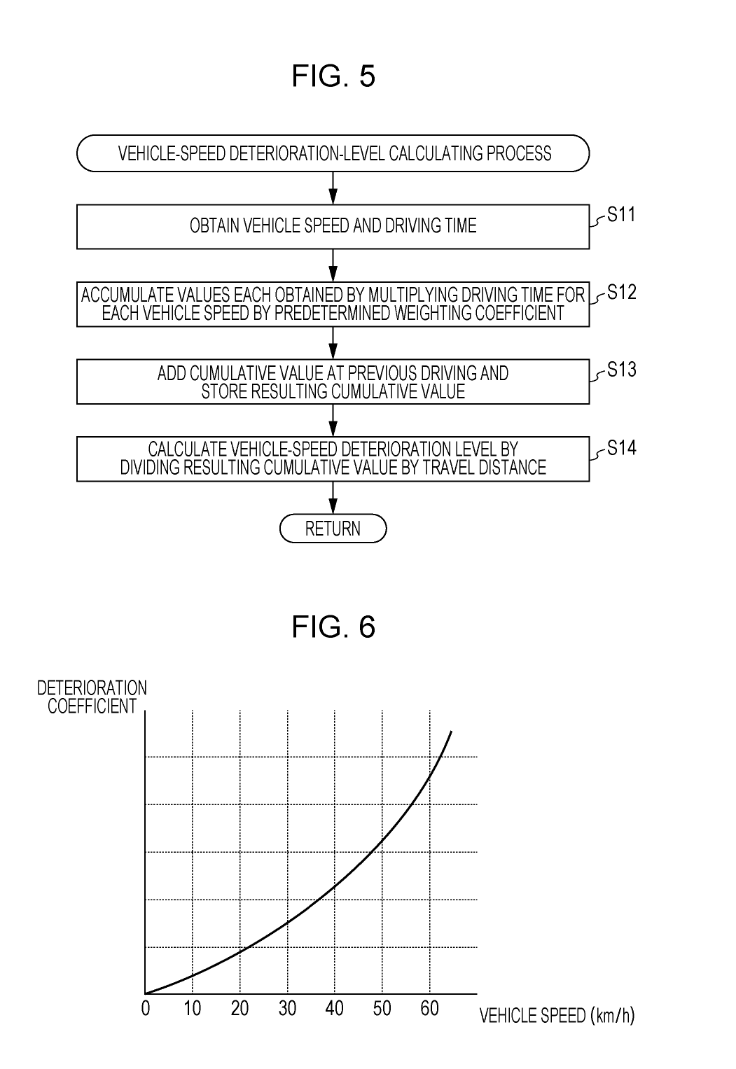

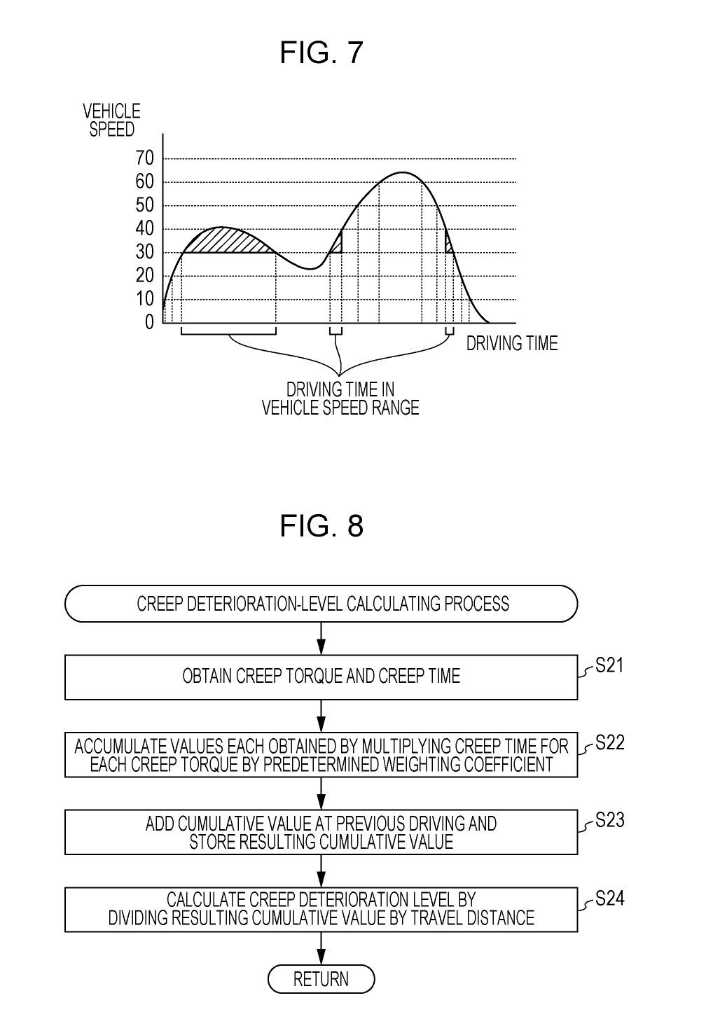

First, a “vehicle-speed deterioration-level calculating process” for calculating a deterioration level related to vehicle speed will be described with reference to FIG. 5 to FIG. 7. FIG. 5 is a flowchart illustrating an embodiment of a vehicle-speed deterioration-level calculating process. FIG. 6 is a characteristic diagram illustrating a relationship between vehicle speed and deterioration coefficient. In FIG. 6, the vertical axis represents deterioration coefficient and the horizontal axis represents vehicle speed. FIG. 7 is a conceptual diagram illustrating driving time in each vehicle speed range.

In step S11, vehicle speed, driving time, and cumulative travel distance are obtained from deterioration element data accumulated in the data accumulating unit 6. In step S12, a vehicle speed frequency (occurrence frequency) is calculated for each vehicle speed range by multiplying, by a predetermined weighting coefficient, a driving time (in seconds) in each predetermined vehicle speed range based on the obtained vehicle speed and driving time. Then, the resulting vehicle speed frequencies for all vehicle speed ranges are accumulated to obtain the sum (cumulative value). The weighting coefficient is determined for each vehicle speed range in accordance with a deterioration coefficient which has a characteristic of increasing quadratically with increasing vehicle speed, as illustrated in FIG. 6. As illustrated in FIG. 7, the obtained vehicle speed is divided into predetermined vehicle speed ranges (e.g., of 10 km/h each). The driving time (in seconds) in each predetermined vehicle speed range is part of the total driving time and is obtained by summing all periods of time during which the vehicle 1 traveled at speeds in this range. In the example of FIG. 7, the driving time for a vehicle speed of 30 km/h to 40 km/h is the sum of periods of driving time indicated by diagonal lines. In step S13, a cumulative value at the previous driving is read from the data accumulating unit 6, the cumulative value obtained in step S12 is added to the read cumulative value, and the resulting cumulative value is stored in the data accumulating unit 6. In step S14, a vehicle-speed deterioration level is calculated by dividing the resulting cumulative value by the cumulative travel distance obtained in step S11.

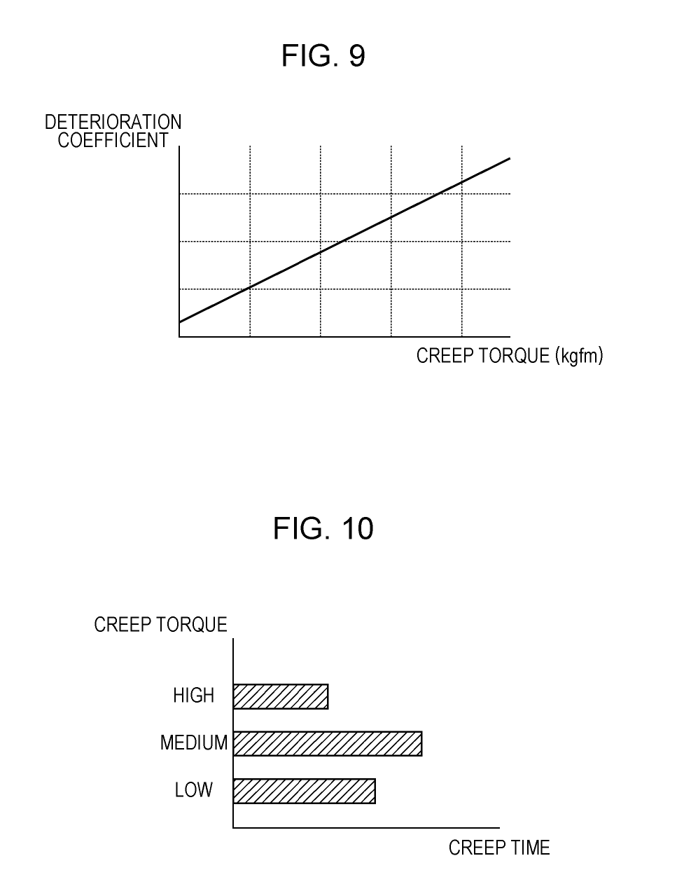

Next, a “creep deterioration-level calculating process” for calculating a deterioration level related to creep torque will be described with reference to FIG. 8 to FIG. 10. FIG. 8 is a flowchart illustrating an embodiment of a creep deterioration-level calculating process. FIG. 9 is a characteristic diagram illustrating a relationship between creep torque and deterioration coefficient. In FIG. 9, the vertical axis represents deterioration coefficient and the horizontal axis represents creep torque. FIG. 10 is a conceptual diagram illustrating creep time for each level of creep torque.

In step S21, creep torque, creep time, and cumulative travel distance are obtained from deterioration element data accumulated in the data accumulating unit 6. In step S22, a creep frequency (occurrence frequency) is calculated for each creep torque level by multiplying, by a predetermined weighting coefficient, a creep time (in seconds) for each predetermined creep torque level based on the obtained creep torque and creep time. Then, the resulting creep frequencies for all creep torque levels are accumulated to obtain the sum (cumulative value). The weighting coefficient is determined for each creep torque level in accordance with a deterioration coefficient which has a characteristic of increasing with increasing creep torque, as illustrated in FIG. 9. As illustrated in FIG. 10, the obtained creep torque is divided into predetermined creep torque levels (e.g., high, medium, and low). The creep time (in seconds) for each creep torque level is part of the total creep time and is obtained by calculating the total length of time during which creep torque of this level was applied. In step S23, a cumulative value at the previous driving is read from the data accumulating unit 6, the cumulative value obtained in step S22 is added to the read cumulative value, and the resulting cumulative value is stored in the data accumulating unit 6. In step S24, a creep deterioration level is calculated by dividing the resulting cumulative value by the cumulative travel distance obtained in step S21.

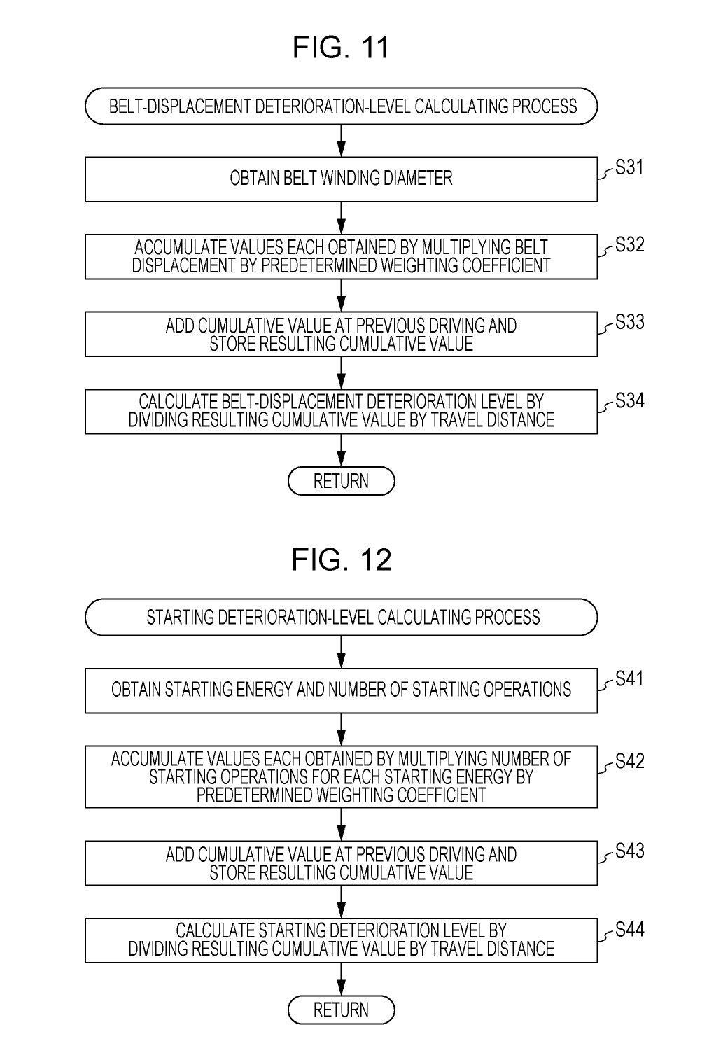

Next, a “belt-displacement deterioration-level calculating process” for calculating a deterioration level related to belt displacement will be described with reference to FIG. 11. FIG. 11 is a flowchart illustrating an embodiment of a belt-displacement deterioration-level calculating process.

In step S31, belt winding diameter and cumulative travel distance are obtained from deterioration element data accumulated in the data accumulating unit 6. In step S32, belt displacement, which is the amount of change from the previous belt winding diameter (at a given gear ratio before gear change), is determined based on the belt winding diameter obtained in step S31. Then, a value (occurrence frequency) obtained by multiplying the belt displacement by a predetermined weighting coefficient is accumulated to obtain the sum (cumulative value). The weighting coefficient is a deterioration coefficient (fixed value) determined in advance for each vehicle type. In step S33, a cumulative value at the previous driving is read from the data accumulating unit 6, the cumulative value obtained in step S32 is added to the read cumulative value, and the resulting cumulative value is stored in the data accumulating unit 6. In step S34, a belt-displacement deterioration level is calculated by dividing the resulting cumulative value by the cumulative travel distance obtained in step S31.

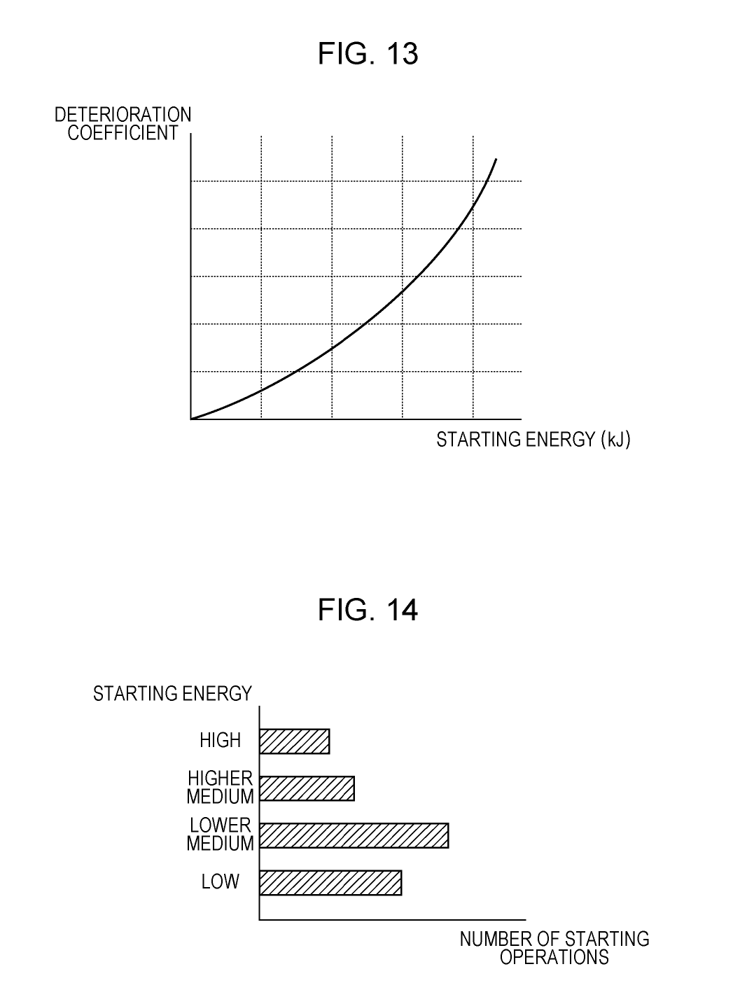

Next, a “starting deterioration-level calculating process” for calculating a deterioration level related to starting energy will be described with reference to FIG. 12 to FIG. 14. FIG. 12 is a flowchart illustrating an embodiment of a starting deterioration-level calculating process. FIG. 13 is a characteristic diagram illustrating a relationship between starting energy and deterioration coefficient. In FIG. 13, the vertical axis represents deterioration coefficient and the horizontal axis represents starting energy. FIG. 14 is a conceptual diagram illustrating the number of starting operations for each level of starting energy.

In step S41, starting energy, the number of starting operations, and cumulative travel distance are obtained from deterioration element data accumulated in the data accumulating unit 6. In step S42, a starting frequency (occurrence frequency) is calculated for each starting energy level by multiplying, by a predetermined weighting coefficient, the number of starting operations for each starting energy level based on the obtained starting energy and the number of starting operations. Then, the resulting starting frequencies for all starting energy levels are accumulated to obtain the sum (cumulative value). The weighting coefficient is determined for each starting energy level in accordance with a deterioration coefficient which has a characteristic of increasing quadratically with increasing starting energy, as illustrated in FIG. 13. As illustrated in FIG. 14, the obtained starting energy is divided into predetermined starting energy levels (e.g., high, higher medium, lower medium, and low). The number of starting operations for each starting energy level is part of the total number of starting operations and is obtained by calculating the total number of times the vehicle 1 was started at this starting energy level. In step S43, a cumulative value at the previous driving is read from the data accumulating unit 6, the cumulative value obtained in step S42 is added to the read cumulative value, and the resulting cumulative value is stored in the data accumulating unit 6. In step S44, a starting deterioration level is calculated by dividing the resulting cumulative value by the cumulative travel distance obtained in step S41.

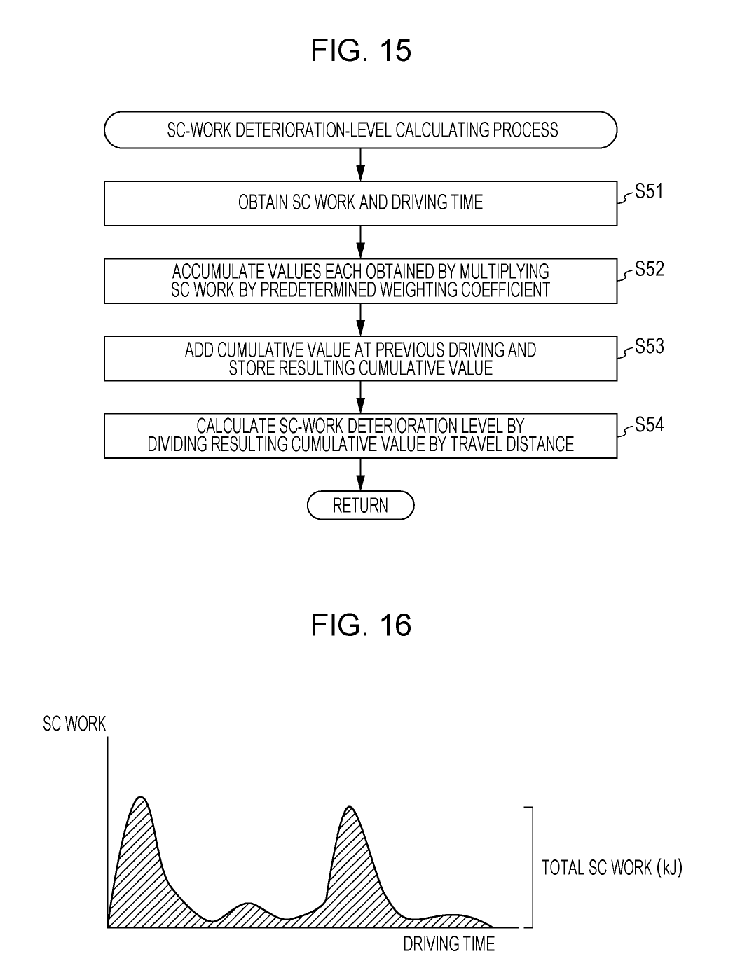

Next, an “SC-work deterioration-level calculating process” for calculating a deterioration level related to SC work will be described with reference to FIG. 15 and FIG. 16. FIG. 15 is a flowchart illustrating an embodiment of an SC-work deterioration-level calculating process. FIG. 16 is a conceptual diagram illustrating total SC work.

In step S51, SC work, driving time, and cumulative travel distance are obtained from deterioration element data accumulated in the data accumulating unit 6. In step S52, a value (occurrence frequency) obtained by multiplying the obtained SC work by a predetermined weighting coefficient is accumulated to obtain the sum (cumulative value). The weighting coefficient is a deterioration coefficient (fixed value) determined in advance for each vehicle type. The cumulative value is the total SC work corresponding to an area indicated by diagonal lines in FIG. 16. In step S53, a cumulative value at the previous driving is read from the data accumulating unit 6, the cumulative value obtained in step S52 is added to the read cumulative value, and the resulting cumulative value is stored in the data accumulating unit 6. In step S54, an SC-work deterioration level is calculated by dividing the resulting cumulative value by the cumulative travel distance obtained in step S51.

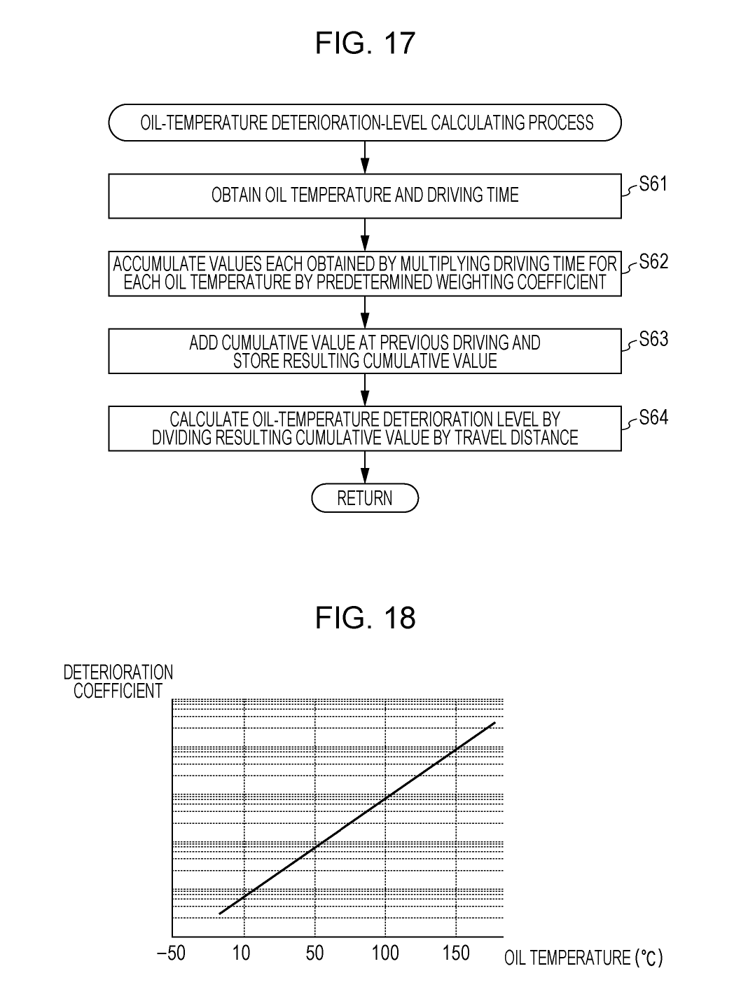

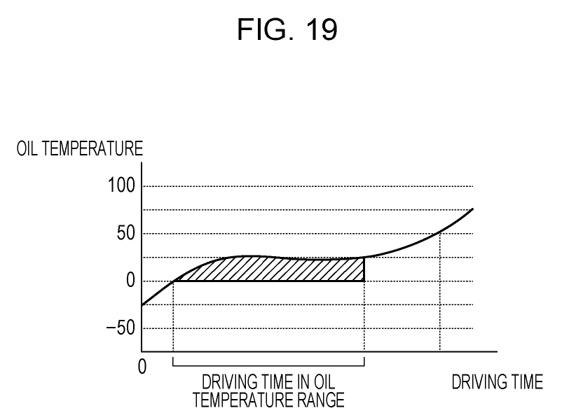

Next, an “oil-temperature deterioration-level calculating process” for calculating a deterioration level related to oil temperature will be described with reference to FIG. 17 to FIG. 19. FIG. 17 is a flowchart illustrating an embodiment of an oil-temperature deterioration-level calculating process. FIG. 18 is a characteristic diagram illustrating a relationship between oil temperature and deterioration coefficient. In FIG. 18, the vertical axis represents deterioration coefficient and the horizontal axis represents oil temperature. FIG. 19 is a conceptual diagram illustrating driving time in each oil temperature range.

In step S61, oil temperature, driving time, and cumulative travel distance are obtained from deterioration element data accumulated in the data accumulating unit 6. In step S62, an oil temperature frequency (occurrence frequency) is calculated for each oil temperature range by multiplying, by a predetermined weighting coefficient, a driving time (in seconds) in each predetermined oil temperature range based on the obtained oil temperature and driving time. Then, the resulting oil temperature frequencies for all oil temperature ranges are accumulated to obtain the sum (cumulative value). The weighting coefficient is determined for each oil temperature range in accordance with a deterioration coefficient which has a characteristic of increasing logarithmically with increasing oil temperature, as illustrated in FIG. 18. As illustrated in FIG. 19, the obtained oil temperature is divided into predetermined oil temperature ranges (e.g., of 25 degrees each). The driving time (in seconds) in each predetermined oil temperature range is part of the total driving time and is obtained by summing all periods of time during which the vehicle 1 traveled at oil temperatures in this range. In step S63, a cumulative value at the previous driving is read from the data accumulating unit 6, the cumulative value obtained in step S62 is added to the read cumulative value, and the resulting cumulative value is stored in the data accumulating unit 6. In step S64, an oil-temperature deterioration level is calculated by dividing the resulting cumulative value by the cumulative travel distance obtained in step S61.

Referring back to FIG. 4, in step S2, a determination is made as to whether the cumulative travel distance of each vehicle 1 is greater than or equal to a predetermined distance. If it is determined that the cumulative travel distance has not reached the predetermined distance (NO in step S2), it is determined that the accumulation of travel distance is insufficient (step S6) and the present process ends. That is, if the cumulative travel distance is insufficient for determining the level of oil deterioration, the way in which the vehicle 1 is used by the user has not yet stabilized (i.e., the amount of cumulative information is too small to be certain about the usage). Therefore, severity of usage of working fluid is not evaluated, and notification of oil change timing is not provided.

On the other hand, if it is determined that the cumulative travel distance has reached the predetermined distance (YES in step S2), a determination is made as to whether any of the deterioration levels for different deterioration factors (deterioration elements) calculated in the “deterioration-level calculating processes for different deterioration factors” described above is greater than or equal to the corresponding threshold value determined for each deterioration factor (step S3). If any of the deterioration levels is greater than or equal to the corresponding threshold value (YES in step S3), the vehicle 1 is evaluated as a “severe user”, which is an index indicating severity of usage of working fluid (step S5). In this case, the vehicle 1 (user) is ranked depending on the type of deterioration level that reaches the threshold value. For example, if the deterioration level that reaches the threshold value is a vehicle-speed deterioration level, the vehicle 1 is ranked as a “severe user A”. Similarly, if a creep deterioration level or a belt-displacement deterioration level reaches the corresponding threshold value, the vehicle 1 is raked as a “severe user B”; if a starting deterioration level or an SC-work deterioration level reaches the corresponding threshold value, the vehicle 1 is ranked as a “severe user C”; and if an oil-temperature deterioration level reaches the corresponding threshold value, the vehicle 1 is raked as a “severe user D”. In other words, each vehicle 1 is ranked depending on the impact of each deterioration factor (deterioration element), such as vehicle speed, starting energy, or oil temperature, on the oil deterioration. For example, a vehicle 1 with a higher-impact deterioration factor is ranked higher. If a plurality of deterioration levels are greater than or equal to their corresponding threshold values, the vehicle 1 is ranked high, such as rank A (or may be ranked low). Thus, each vehicle 1 is ranked on a scale of “severe user A” to “severe user D” or the like, depending on the severity of usage of working fluid.

If it is determined that none of the deterioration levels reaches the corresponding threshold value (NO in step S3), the vehicle 1 is evaluated as a “normal user”, which is also an index indicating severity of usage of working fluid (step S4). In step S7, oil change timing is determined in accordance with the evaluation described above. In step S8, notification of the determined oil change timing is transmitted to the vehicle 1. The oil change timing varies depending on the rank in the evaluation. If the vehicle 1 (user) is ranked higher, the severity of usage of working fluid of the vehicle 1 is higher than that of the others ranked lower. This means that the higher the rank of the vehicle 1, the more it is necessary to shorten the oil change cycle. When an evaluation such as that described above is performed, oil change timing can be determined, for example, by estimating how oil deterioration will proceed if the vehicle 1 continues to be driven in the same manner as in the past driving records (e.g., travel distance and driving time) based on the deterioration level (i.e., “oil deterioration estimate per unit distance”, driving time, etc.), or by performing inverse calculation from a change cycle (time period or distance) determined in advance for each rank. If the vehicle 1 is evaluated as a “normal user”, the oil change timing may be determined by inverse calculation in accordance with an oil change criterion, such as a predetermined cumulative travel distance of the vehicle 1 or elapsed time, which serves as a certain measure for determining when to change oil.

As described above, the vehicle working-fluid evaluating system according to the embodiment of the present invention monitors usage of a vehicle by obtaining a plurality of deterioration elements related to various deterioration factors that occur as the vehicle travels and can accelerate deterioration of working fluid. In accordance with oil deterioration estimates calculated based on the plurality of deterioration elements obtained, the vehicle working-fluid evaluating system determines severity of usage of the working fluid for a transmission mounted on the vehicle. In accordance with the severity of usage of the working fluid, the vehicle working-fluid evaluating system determines timing of when to change the working fluid and notifies the vehicle of the timing. The oil deterioration estimates correspond to the levels of deterioration of working fluid determined based on how the vehicle has been used. With the oil deterioration estimates, it is possible to estimate damage the working fluid may suffer if the vehicle continues to be used in the same manner as it was. Therefore, for a deterioration element related to a deterioration factor that causes severe deterioration of working fluid, the severity of usage of working fluid is evaluated as being high. Then, the oil change timing is determined such that the higher the severity evaluation, the shorter the oil change cycle. Thus, by evaluating the severity of usage of working fluid based on various deterioration factors which may accelerate deterioration of the working fluid, it is possible to estimate the future progress of deterioration of the working fluid. Therefore, it is possible to determine appropriate oil change timing before deterioration of working fluid and notify each vehicle (user) in advance of the oil change timing, which may vary from one vehicle to another.

Although some embodiments have been described with reference to the drawings, the present invention is not limited to them. It is to be understood that various other embodiments are possible. For example, in the embodiments described above, a continuously variable transmission has been described as an example of the automatic transmission for which severity of usage of working fluid is to be evaluated. However, the automatic transmission is not limited to the continuously variable transmission. A transmission for which severity of usage of working fluid is to be evaluated may not even be an automatic transmission.

Although each vehicle (or driver) is notified of oil change timing in the embodiments described above, the present invention is not limited to this. Notification of oil change timing for all vehicles registered in advance may be provided to user terminals which are not mounted on the vehicles and are owned by various persons who are not drivers but are related to the vehicles, such as mechanics or dealers.

Any of the plurality of deterioration elements described above may be specified for which the “oil change notification process” is to be performed. This is convenient in that, together with notification of oil change timing, it is possible to notify that conditions of related parts (e.g., the tension of the metal V-belt and the wear of pulleys) are to be checked.

According to the embodiment of the present invention, the vehicle working-fluid evaluating system monitors usage of the vehicle 1 by obtaining a plurality of deterioration elements related to various deterioration factors that occur as the vehicle 1 travels and can accelerate deterioration of working fluid. In accordance with oil deterioration estimates calculated based on the plurality of deterioration elements obtained, the vehicle working-fluid evaluating system determines severity of usage of the working fluid for the transmission CVT mounted on the vehicle 1. In accordance with the severity of usage of the working fluid, the vehicle working-fluid evaluating system determines timing of when to change the working fluid and notifies the vehicle 1 of the timing. The oil deterioration estimates correspond to the levels of deterioration of working fluid determined based on how the vehicle has been used. With the oil deterioration estimates, it is possible to estimate damage the working fluid may suffer if the vehicle continues to be used in the same manner as it was. Therefore, for a deterioration element related to a deterioration factor that causes severe deterioration of working fluid, the severity of usage of working fluid is evaluated as being high. Then, the oil change timing is determined such that the higher the severity evaluation, the shorter the oil change cycle. Thus, by evaluating the severity of usage of working fluid based on various deterioration factors which may accelerate deterioration of the working fluid, it is possible to estimate the future progress of deterioration of the working fluid. Therefore, it is possible to determine appropriate oil change timing before deterioration of working fluid and notify each vehicle (user) in advance of the oil change timing, which may vary from one vehicle to another.

Obviously, numerous modifications and variations of the present invention are possible in light of the above teachings. It is therefore to be understood that within the scope of the appended claims, the invention may be practiced otherwise than as specifically described herein.

What is claimed is:

1. A vehicle working-fluid evaluating system to indicate working-fluid change timing when working fluid for a transmission mounted on a vehicle is changed, the vehicle working-fluid evaluating system comprising:
an obtaining device configured to obtain a plurality of deterioration elements related to deterioration factors that are generated as the vehicle travels and that accelerate deterioration of the working fluid;
a deterioration-level calculator configured to calculate, based on the plurality of deterioration elements obtained by the obtaining device, an oil deterioration estimate corresponding to a level of deterioration of the working fluid for each of the plurality of deterioration elements;
a severity determining device configured to determine, in accordance with the oil deterioration estimate calculated by the deterioration-level calculator, severity of usage of the working fluid for the transmission mounted on the vehicle;
a change-timing determining device configured to determine the working-fluid change timing in accordance with the severity of usage of the working fluid determined by the severity determining device; and
a notification device configured to indicate the determined working-fluid change timing determined by the change-timing determining device.

2. The working-fluid evaluating system according to claim 1,
wherein the deterioration-level calculator accumulates occurrence frequencies each being determined from the plurality of deterioration elements obtained by the obtaining device and a predetermined deterioration coefficient determined depending on the plurality of deterioration elements, and the deterioration-level calculator divides a resulting cumulative value accumulated by the deterioration-level calculator by a travel distance to calculate the oil deterioration estimate indicating a level of deterioration of the working fluid per unit distance.

3. The working-fluid evaluating system according to claim 1,
wherein the transmission mounted on the vehicle is a continuously variable transmission, and
wherein the plurality of deterioration elements include at least one of vehicle speed, creep force, starting energy, oil temperature, a displacement amount of a belt specific to the continuously variable transmission, and a work amount of a start clutch specific to the continuously variable transmission.

4. The working-fluid evaluating system according to claim 2,
wherein the transmission mounted on the vehicle is a continuously variable transmission, and
wherein the plurality of deterioration elements include at least one of vehicle speed, creep force, starting energy, oil temperature, a displacement amount of a belt specific to the continuously variable transmission, and a work amount of a start clutch specific to the continuously variable transmission.
